SEQUENCE LISTING (1 sequence)

SEQ ID NO: 1 - DNA, 1429 bp, Lactobacillus reuteri
aatggttaggccaccgactttgggcgttacaaactcccatggtgtgacgggcggtgtgtacaaggcccgggaacgtattcaccgcggcatgctgatccgcgattactagcgattccgacttcgtgtaggcgagttgcagcctacagtccgaactgagaacggctttaagagattagcttactctcgcgagcttgcgactcgttgtaccgtccattgtagcacgtgtgtagcccaggtcataaggggcatgatgatctgacgtcgtccccaccttcctccggtttgtcaccggcagtctcactagagtgcccaacttaatgctggcaactagtaacaagggttgcgctcgttgcgggacttaacccaacatctcacgacacgagctgacgacgaccatgcaccacctgtcattgcgtccccgaagggaacgccttatctctaaggttagcgcaagatgtcaagacctggtaaggttcttcgcgtagcttcgaattaaaccacatgctccaccgcttgtgcgggcccccgtcaattcctttgagtttcaaccttgcggtcgtactccccaggcggagtgcttaatgcgttagctccgggcactgaagggcggaaaccctccaacacctagcactcatcgtttacggcatggactaccagggtatctaatcctgttcgctacccatgctttcgagcctcagcgtcagttgcagaccagacagccgccttcgccactggtgttcttccatatatctacgcattccaccgctacacatggagttccactgtcctcttctgcactcaagtcgcccggtttccgatgcacttcttcggttaagccgaaggctttcacatcagacctaagcaaccgcctgcgctcgctttacgcccaataaatccggataacgcttgccacctacgtattaccgcggctgctggcacgtagttagccgtgactttctggttggataccgtcactgcgtgaacagttactctcacgcacgttcttctccaacaacagagctttacgagccgaaacccttcttcactcacgcggtgttgctccatcaggcttgcgcccattgtggaagattccctactgctgcctcccgtaggagtatggaccgtgtctcagttccattgtggccgatcagtctctcaactcggctatgcatcatcgccttggtaagccgttaccttaccaactagctaatgcaccgcaggtccatcccagagtgatagccaaagccatctttcaaacaaaagccatgtggcttttgttgttatgcggtattagcatctgtttccaaatgttatcccccgctccggggcaggttacctacgtgttactcacccgtccgccactcactggtgatccatcgtcaatcaggtgcaagcaccatcaatcagttgggccagtgcgtacgactgc

(19) United States
(12) Patent Application Publication
(10) Pub. No.: US 2025/0163365 A1
(43) Pub. Date: May 22, 2025

(54) LACTOBACILLUS REUTERI JH53 STRAIN HAVING EXCELLENT ORGANIC ACID RESISTANCE AND CONVERSION OF 1,3-PROPANDIOL FROM GLYCEROL, AND USE THEREOF

(71) Applicant: ACTIVON CO., LTD., Chungcheongbuk-do (KR)

(72) Inventors: Chul Ho KIM, Daejeon (KR); Baek Rock OH, Daejeon (KR); Sun-Yeon HEO, Daejeon (KR); Jung-Hyun JU, Daejeon (KR)

(73) Assignee: ACTIVON CO., LTD., Chungcheongbuk-do (KR)

(21) Appl. No.: 18/287,504

(22) PCT Filed: Apr. 14, 2022
(86) PCT No.: PCT/KR2022/005399

§ 371 Date: Jan. 27, 2025

(30) Foreign Application Priority Data
Apr. 20, 2021 (KR) 10-2021-0051173

(51) Int. Cl. C12N 1/20; A61K 8/99; A61Q 19/00; C12P 7/18; C12R 1/225

(52) U.S. Cl. CPC C12N 1/205; A61K 8/99; A61Q 19/00; C12P 7/18; C12R 2001/225

(57) ABSTRACT

The present invention relates to a Lactobacillus reuteri JH53 strain with excellent organic acid resistance and conversion rate of glycerol to 1,3-propanediol, and the use thereof. The strain of the present invention has excellent organic acid resistance and conversion rate of glycerol to 1,3-propanediol compared to the parent strain. Thus, if the strain of the present invention, which is generally recognized as safe (GRAS) for humans, is used in the process of producing 1,3-propanediol from crude glycerol, it may have the advantages of being safe, being environmentally friendly, and recycling resources, unlike existing GMO strains, and thus will be very useful in related industries.

### TECHNICAL FIELD

The present invention relates to a Lactobacillus reuteri JH53 strain with excellent organic acid resistance and conversion rate of glycerol to 1,3-propanediol, and the use thereof.

### BACKGROUND ART

Fermentation refers to a metabolic process in which microorganisms obtain energy by breaking down sugars under oxygen-free conditions. Pyruvic acid produced through glycolysis reacts with NADH to produce NAD+ and organic products. At this time, the produced organic products include organic acids such as lactic acid, acetic acid, butyric acid, carboxylic acid, and citric acid, as well as ethanol, carbon dioxide, hydrogen, and the like. Fermentation generally occurs in an anaerobic environment, because, in the presence of oxygen, pyruvic acid and NADH are used to generate ATP through aerobic respiration. Most industrial fermentations are carried out through batch culture or fed-batch culture methods. Batch culture is a general method of carrying out the fermentation reaction without additional nutrient supply until the end of the culture. Fed-batch culture is a method that can more effectively control each step of the fermentation process by continuously supplying substrates.

Lactobacillus species which exist in large quantities in fermented foods such as kimchi and cheese, are Gram-positive bacteria that obtain energy through the fermentation process and produce a large amount of lactic acid in the process. Although Lactobacillus species are known as beneficial bacteria that induce the death of harmful bacteria by lactic acid produced as a result of fermentation, they die without growing if fermentation continues and the surrounding acidity becomes excessively high due to the lactic acid secreted by them.

Meanwhile, crude glycerol is generated as a main by-product of the current biodiesel production process, which accounts for about 10% (w/w) of total biodiesel production. This crude glycerol not only affects the price of glycerol in the existing traditional glycerol market, but also contains salts, unreacted fatty acids and a large amount of impurities, and thus cannot be released directly into the environment. Therefore, research has been actively conducted on methods of producing industrially valuable materials by converting inexpensive crude glycerol into biofuel or biologically active materials.

Glycerol can be converted through microbial fermentation into various chemical raw materials, a representative example of which is 1,3-propanediol. 1,3-Propanediol is used in fibers such as high-performance clothing, carpets, and automobile fabrics, as well as plastic films, cosmetics, and the like. In particular, 1,3-propanediol is used as a cosmetic raw material called “propanediol” and is known to have excellent moisturizing and preservative effects compared to 1,3-butylene glycol, a similar substance. Propanediol is classified as Grade 1, the highest safety grade, by the Environmental Working Group (EWG), an American environmental research group, and has a similar level of moisturizing ability to glycerin, but is known to have excellent skin absorption ability due to its low viscosity. In addition, propanediol is safe as it has little skin irritation, and can also act as a preservative, and thus it is widely used as a cosmetic and food additive. However, since 1,3-propanediol is currently mostly produced by recombinant strains (E. coli, Klebsiella or Clostridium), it is highly rejected in the market due to GMO issues.

Meanwhile, Korean Patent No. 1951919 discloses ‘a novel Lactobacillus reuteri ATG-F4 strain with the function of enhancing dopamine secretion and a composition for preventing or treating mental disorders containing the same’, and Korean Patent No. 1655853 discloses ‘a Lactobacillus reuteri CBG-C15 strain producing conjugated linoleic acid and the use thereof’. However, there is no disclosure regarding ‘a Lactobacillus reuteri JH53 strain with excellent organic acid resistance and conversion rate of glycerol to 1,3-propanediol and the use thereof’ according to the present invention.

### DISCLOSURE

### Technical Problem

The present invention has been made according to the above requirements. The present inventors induced mutations in the parent strain Lactobacillus reuteri CH53, which highly produces 1,3-propanediol from glycerol, by electron beam irradiation, and then selected mutant strains with increased organic acid resistance compared to the CH53 strain. Thereamong, a Lactobacillus reuteri JH53 strain having increased production of 1,3-propanediol compared to the parent strain has been finally selected and subjected to fed-batch culture with continuous feeding of glycerol. As a result, the present inventors found that cell growth of the Lactobacillus reuteri JH53 strain of the present invention continued longer than that of the parent strain, and the production of 1,3-propanediol increased by about 34.6%, thereby completing the present invention.

### Technical Solution

To solve the above problem, the present invention provides a Lactobacillus reuteri JH53 strain (accession number: KCTC14360BP) with excellent organic acid resistance and conversion rate of glycerol to 1,3-propanediol.

The present invention also provides a composition for producing 1,3-propanediol containing the above strain or a culture thereof as an active ingredient.

The present invention also provides a cosmetic composition for moisturizing skin containing the above strain or a culture thereof as an active ingredient.

The present invention also provides a method for producing 1,3-propanediol comprising a step of culturing the above strain.

### Advantageous Effects

The Lactobacillus reuteri JH53 strain of the present invention has excellent organic acid resistance and conversion rate of glycerol to 1,3-propanediol compared to the parent strain. Thus, if the strain of the present invention, which is generally recognized as safe (GRAS) for humans, is used in the process of producing 1,3-propanediol from crude glycerol, it may have the advantages of being safe, being environmentally friendly, and recycling resources, unlike existing GMO strains, and thus will be very useful in related industries.

### BRIEF DESCRIPTION OF DRAWINGS

FIG. 1 shows the results of measuring the production of 1,3-propanediol while culturing the Lactobacillus reuteri CH53 strain in a medium containing lactic acid or acetic acid.

FIG. 2 shows the results of culturing the Lactobacillus reuteri CH53 strain in media containing mixed organic acids at different concentrations.

FIG. 3 shows the results of fed-batch culture of the Lactobacillus reuteri CH53 strain (A) and the Lactobacillus reuteri JH53 strain (B).

### BEST MODE

To achieve the object of the present invention, the present invention provides a Lactobacillus reuteri JH53 strain (accession number: KCTC14360BP) with excellent organic acid resistance and conversion rate of glycerol to 1,3-propanediol.

In the present invention, a Lactobacillus reuteri CH53 strain (accession number: KCTC13149BP), which highly produces 1,3-propanediol from glycerol, was used as a parent strain, mutations therein were induced by electron beam irradiation, and then a Lactobacillus reuteri JH53 strain with excellent organic acid resistance and conversion rate of glycerol to 1,3-propanediol compared to the parent strain was finally selected.

As a result of fed-batch culturing the Lactobacillus reuteri JH53 strain of the present invention and the parent strain Lactobacillus reuteri CH53 for 72 hours under the same conditions, it was found that the parent strain reached the death phase after 24 hours of culture, whereas cell growth of the Lactobacillus reuteri JH53 strain of the present invention did not decrease until 72 hours of culture, and lactic acid production of the Lactobacillus reuteri JH53 strain decreased compared to that of the parent strain. In addition, it was found that 1,3-propanediol production of the Lactobacillus reuteri JH53 strain increased compared to that of the parent strain. Accordingly, the present inventors named the mutant strain as Lactobacillus reuteri JH53 strain and deposited the same with the KCTC at the Korea Research Institute of Bioscience and Biotechnology on Nov. 9, 2020 (accession number: KCTC14360BP). The Lactobacillus reuteri JH53 strain may comprise 16s rRNA consisting of the nucleotide sequence represented by SEQ ID NO: 1.

The Lactobacillus reuteri JH53 strain (KCTC14360BP) of the present invention is characterized by exhibiting a production yield of 0.814 g/g (1,3-propanediol/glycerol), which is 98% relative to the theoretical yield (0.83 g/g) and corresponds to a very high conversion rate.

In the present invention, the conversion rate to 1,3-propanediol is a value expressed as the percentage of the actual production yield of 1,3-propanediol, determined per amount of glycerol added to a medium for culturing the Lactobacillus reuteri JH53 strain of the present invention, relative to the theoretical production yield of 1,3-propanediol (0.83 g/g).

In addition, the Lactobacillus reuteri JH53 strain (KCTC14360BP) of the present invention is characterized in that 1,3-propanediol production of the strain increased by 30 to 40% compared to that of the parent strain Lactobacillus reuteri CH53 strain (KCTC13149BP).

In the present invention, the organic acid may be lactic acid or acetic acid, without being limited thereto.

The present invention also provides a composition for producing 1,3-propanediol containing the above-described strain or a culture thereof as an active ingredient.

In the composition according to one embodiment of the present invention, the strain is preferably the Lactobacillus reuteri JH53 strain (accession number: KCTC14360BP), without being limited thereto.

The present invention also provides a cosmetic composition for moisturizing skin containing the above-described strain or a culture thereof as an active ingredient.

In the cosmetic composition of the present invention, the strain is preferably the Lactobacillus reuteri JH53 strain (accession number: KCTC14360BP), and the culture thereof is preferably in the form of lyophilized powder, without being limited thereto.

The cosmetic composition of the present invention may be provided in the form of solution, gel, solid or pasty anhydrous product, emulsion obtained by dispersing oil phase in water phase, suspension, microemulsion, microcapsule, microgranule or ionic liposome, nonionic vesicle dispersion, cream, skin, lotion, powder, ointment, essence, spray, or conceal stick. In addition, the cosmetic composition of the present invention may also be prepared as a foam composition or an aerosol composition further containing a compressed propellant.

In addition, the cosmetic composition of the present invention may contain, in addition to the strain of the present invention or a culture thereof, an auxiliary commonly used in the cosmetic field, such as a fatty substance, an organic solvent, a solubilizer, a thickener, a gelling agent, a softener, an antioxidant, a suspending agent, a stabilizer, a foaming agent, a flavoring agent, a surfactant, water, an ionic or nonionic emulsifying agent, a filler, a metal ion sequestering agent, a chelating agent, a preservative, a vitamin, a blocking agent, a wetting agent, an essential oil, a dye, a pigment, a hydrophilic or lipophilic activator, a lipid vesicle, or any other ingredient commonly used in cosmetics.

The cosmetic composition for skin moisturizing according to the present invention may further contain a known skin-moisturizing ingredient in addition to the above-described strain or a culture thereof. When an additional ingredient helpful for skin health is contained, the effect of the cosmetic composition for moisturizing skin according to the present invention may be further improved, and when an ingredient helpful for skin health is added, skin safety, ease of formulation, and stability of active ingredients may be considered in relation to the use of this ingredient.

The present invention also provides a method for producing 1,3-propanediol comprising a step of culturing the above-described strain.

In the production method of the present invention, the strain is preferably a Lactobacillus reuteri JH53 strain (accession number: KCTC14360BP), without being limited thereto.

In addition, the method for producing 1,3-propanediol according to the present invention may comprise culturing the strain in a medium containing glycerol.

The method for producing 1,3-propanediol according to the present invention may further comprise a step of recovering 1,3-propanediol from the medium in which the strain has been cultured. The step of recovering 1,3-propanediol from the medium in which the strain has been cultured may be performed using conventional isolation techniques, such as distillation, electrodialysis, pervaporation, chromatography, solvent extraction, reaction extraction, etc. These techniques may be used in combination to separate substances of high purity.

The method of culturing the strain of the present invention may be performed according to a culturing method commonly used in the art, and is not limited to a particular method.

Hereinafter, the present invention will be described in detail with reference to examples. However, the following examples are intended to only illustrate the present invention, and the scope of the present invention is not limited thereto.

### Example 1. Analysis of Organic Acid Resistance of Lactobacillus reuteri CH53 Strain

Lactobacillus species use glucose as a nutritional and energy source for cell growth. When metabolites such as lactic acid, acetic acid, and ethanol produced during the oxidation of glucose accumulate, they can inhibit the growth of cells and reduce the production of useful substances.

### (1) Measurement of Half-Maximal Inhibitory Concentrations (IC₅₀) for Organic Acids

The half-maximal inhibitory concentrations (IC₅₀) of the Lactobacillus reuteri CH53 strain (KCTC13149BP) for major metabolites were measured. Each of 1,3-propanediol, ethanol, lactic acid, and acetic acid was added at a concentration of 0 to 100 g/L to MRS broth containing 10 g/L of proteose peptone NO. 3, 10 g/L of beef extract, 5 g/L of yeast extract, 20 g/L of glucose, 1 g/L of polysorbate 80, 2 g/L of ammonium citrate, 5 g/L of sodium acetate, 0.1 g/L of magnesium sulfate, 0.05 g/L of manganese sulfate, and 2 g/L dipotassium phosphate, and the pH was adjusted to 5.5. Thereafter, 1% (v/v) of the strain culture with OD_{600 nm}=8.0 was inoculated into each broth and statically cultured at 37° C. for 24 hours, and the concentration at which the absorbance (OD_{600 nm}) was half of the control was measured.

As a result, it was found that the IC₅₀ values of the Lactobacillus reuteri CH53 strain for 1,3-propanediol, ethanol, lactic acid, and acetic acid were 79.8 g/L, 29.5 g/L, 27.9 g/L, and 17.4 g/L, respectively (Table 1).

TABLE 1
IC₅₀ values of Lactobacillus reuteri CH53 strain for metabolites

| Metabolite | IC₅₀ (g/L) |
|---|---|
| 1,3-propanediol | 79.8 |
| Ethanol | 29.5 |
| Lactic acid | 27.9 |
| Acetic acid | 17.4 |

(2) Analysis of Cell Growth and 1,3-Propanediol Production in Medium Supplemented with Organic Acid

Cell growth and 1,3-propanediol production were measured while the Lactobacillus reuteri CH53 strain was fed-batch cultured in a medium supplemented with an organic acid. 3 L of MRS broth containing 20 g/L of glycerol, 10 g/L of proteose peptone NO. 3, 10 g/L of beef extract, 5 g/L of yeast extract, 20 g/L of glucose, 1 g/L of polysorbate 80, 2 g/L of ammonium citrate, 5 g/L of sodium acetate, 0.1 g/L of magnesium sulfate, 0.05 g/L of manganese sulfate, and 2 g/L dipotassium phosphate was placed in a 5-L fermenter, and 10 g/L of lactic acid or 10 g/L of acetic acid was added thereto, and the pH was adjusted to 5.5. During culture, glucose and glycerol were continuously fed so that they could be maintained at residual concentrations of 5 to 10 g/L. Thereafter, 1% (v/v) of the strain culture with OD_{600 nm}=8.0 was inoculated into each broth, and the absorbance (OD_{600 nm}) was measured while fed-batch culture was carried out under conditions of 37° C. and 100 rpm. 1 mL of each culture was centrifuged at 13,000 rpm for 5 minutes, and then the supernatant was filtered through a 0.22-μm filter and the concentration of 1,3-propanediol was measured using high-performance liquid chromatography. 1,3-Propanediol production was analyzed by high-performance liquid chromatography equipped with an ion exchange column (300×78 mm, Aminex HPX-87H, Bio-Rad) and a differential refraction detector (RID) using 0.25 mM H₂SO₄ as a mobile phase at a flow rate of 0.6 ml/min.

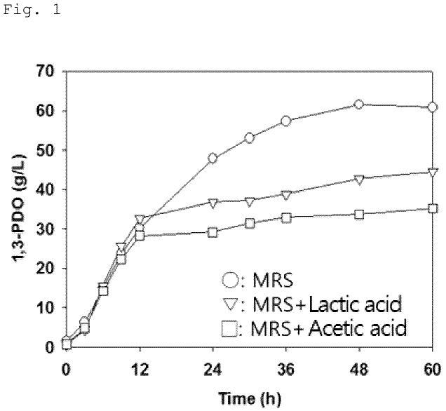

As a result, it was found that lactic acid or acetic acid did not affect cell growth of the Lactobacillus reuteri CH53 strain, but reduced 1,3-propanediol production (FIG. 1).

### Example 2. Development of Mutant Strain with Enhanced Organic Acid Resistance

Since organic acids inhibit 1,3-propanediol production of the Lactobacillus reuteri CH53 strain (accession number: KCTC13149BP), random mutations in the strain were induced by electron beam irradiation, a traditional strain improvement technique, and then mutant strains with enhanced organic acid resistance were selected.

### (1) Establishment of Mutant Strain Selection Conditions

In order to establish conditions for selecting mutant strains with enhanced organic acid resistance, the concentration of the Lactobacillus reuteri CH53 strain, at which it is resistant to mixed organic acids, was investigated. 15 g/L of agar was added to MRS broth containing 10 g/L of proteose peptone NO. 3, 10 g/L of beef extract, 5 g/L of yeast extract, 20 g/L of glucose, 1 g/L of polysorbate 80, 2 g/L of ammonium citrate, 5 g/L of sodium acetate, 0.1 g/L of magnesium sulfate, 0.05 g/L of manganese sulfate, and 2 g/L dipotassium phosphate, and lactic acid and acetic acid were added thereto under the conditions shown in Table 2 below, thereby preparing plate media (#1 to #6) containing mixed organic acids. Thereafter, the Lactobacillus reuteri CH53 strain was streaked on each plate medium and statically cultured in an anaerobic chamber at 37° C. for 24 hours.

TABLE 2
Concentrations of mixed organic acids for establishing
organic acid-resistant strain selection conditions

| Plate medium containing mixed | Lactic acid | Acetic acid |
|---|---|---|
| organic acids | (g/L) | (g/L) |
| #1 | 20.0 | 10.0 |
| #2 | 25.0 | 12.5 |
| #3 | 30.0 | 15.0 |
| #4 | 35.0 | 17.5 |
| #5 | 40.0 | 20.0 |
| #6 | 45.0 | 22.5 |

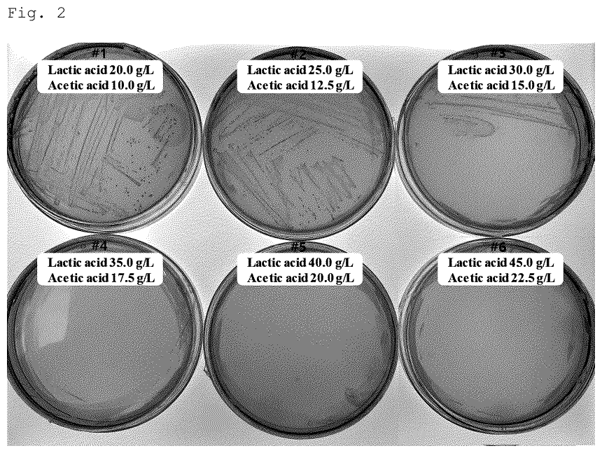

As a result, it was found that the Lactobacillus reuteri CH53 strain could not grow at a lactic acid concentration of 35.0 g/L or higher and an acetic acid concentration of 17.5 g/L or higher (FIG. 2).

### (2) Mutant Strain Production

Mutant strains with enhanced organic acid resistance were produced using an electron beam irradiation mutagenesis method. The Lactobacillus reuteri CH53 strain was inoculated into 50-ml MRS broth containing 20 g/L glycerol, and statically cultured at 37° C. for 8 hours, followed by centrifugation at 4,000 rpm and 4° C. Next, the sample was washed once with sterile PBS buffer (pH 5.5) and resuspended in 10 mL of sterile PBS buffer (pH 5.5). Electron beam irradiation was carried out at the Korea Atomic Energy Research Institute (Jeongeup, Jeonbuk, Korea), using a 10-MeV linear electron accelerator (MB10-30; Mevex Corp, Canada). The under beam conveyor speed was 2.100 to 3.272 m/min, and the dose applied was about 1.0 to 3.0 kGy. After electron beam irradiation, 10 ml of MRS medium was added to the sample which was then statically cultured in an anaerobic chamber at 37° C. for 4 hours. Next, the sample was spread on an MRS plate medium containing 35.0 g/L of lactic acid and 17.5 g/L of acetic acid, which were the established mutant strain selection conditions, and statically cultured in an anaerobic chamber at 37° C. for 72 hours to obtain colonies. Next, the colonies were subcultured three times on an MRS plate medium containing 35.0 g/L of lactic acid and 17.5 g/L of acetic acid, and three mutant strains (JH20, JH22, and JH53) with enhanced organic acid resistance compared to the parent strain were obtained.

### (3) Final Selection of Mutant Strain

1,3-Propanediol production and organic acid resistance were compared and analyzed through batch culture of the parent strain (CH53) and three mutant strains with enhanced organic acid resistance (JH20, JH22, and JH53). Each of the Lactobacillus reuteri CH53 strain, Lactobacillus reuteri JH20, Lactobacillus reuteri JH22, and Lactobacillus reuteri JH53 was inoculated into MRS broth containing 20 g/L of glycerol, and statically cultured in an anaerobic chamber at 37° C. for 12 hours. Thereafter, glucose consumption, glycerol consumption, 1,3-propanediol production, and cell growth were measured.

As a result, it was found that, among the mutant strains, the Lactobacillus reuteri JH20 and JH22 strains had reduced glycerol consumption and 1,3-propanediol production compared to the parent strain, whereas the Lactobacillus reuteri JH53 strain maintained cell growth and 1,3-propanediol production similar to those of the parent strain (Table 3).

TABLE 3
Results of batch culture of parent strain and mutant strains

| | | | | 1,3- | |
|---|---|---|---|---|---|
| | | Glucose | Glycerol | propanediol | Cell |
| | | consumption | consumption | production | growth |
| Strain | | (g/L) | (g/L) | (g/L) | (OD_{600 nm}) |
| Parent | L. reuteri CH53 | 20.0 | 15.2 | 12.8 | 9.8 |
| strain | | | | | |
| Mutant | L. reuteri JH20 | 19.9 | 14.1 | 11.8 | 9.7 |
| strain | L. reuteri JH22 | 20.0 | 14.2 | 11.7 | 9.5 |
| | L. reuteri JH53 | 19.8 | 15.1 | 12.7 | 9.8 |

Based on the above results, among the mutant strains with enhanced organic acid resistance compared to the parent strain, a strain with 1,3-propanediol production similar to that of the parent strain was finally selected and named Lactobacillus reuteri JH53.

### Example 3. Characterization of Finally Selected Mutant Strain

### (1) Confirmation of Enhanced Organic Acid Resistance

In order to confirm that the Lactobacillus reuteri JH53 strain of the present invention has enhanced organic acid resistance, the half-maximal inhibitory concentrations (IC₅₀) for organic acids were measured. Each of lactic acid and acetic acid was added at a concentration of 0 to 100 g/L to MRS broth containing 10 g/L of proteose peptone NO. 3, 10 g/L of beef extract, 5 g/L of yeast extract, 20 g/L of glucose, 1 g/L of polysorbate 80, 2 g/L of ammonium citrate, 5 g/L of sodium acetate, 0.1 g/L of magnesium sulfate, 0.05 g/L of manganese sulfate, and 2 g/L dipotassium phosphate, and the pH was adjusted to 5.5. Thereafter, 1% (v/v) of each strain culture with OD_{600 nm}=8.0 was inoculated into each broth and statically cultured at 37° C. for 24 hours, and the concentration at which the absorbance (OD_{600 nm}) was half of the control was measured.

As a result, it was found that the IC₅₀ values of the Lactobacillus reuteri JH53 strain for lactic acid and acetic acid increased by 15.0% and 24.3%, respectively, compared to the parent strain Lactobacillus reuteri CH53 (Table 4).

TABLE 4
Confirmation of enhanced organic acid resistance

| | IC₅₀ (g/L) | | Increase rate |
|---|---|---|---|
| Organic acid | L. reuteri CH53 | L. reuteri JH53 | (%) |
| Lactic acid | 31.9 | 36.7 | 15.0 |
| Acetic acid | 20.6 | 25.6 | 24.3 |
| Total organic acid | 52.5 | 62.3 | 18.7 |

### (2) Confirmation of Increased 1,3-Propanediol Production

In order to compare and analyze 1,3-propanediol production of the Lactobacillus reuteri JH53 strain of the present invention with that of the parent strain Lactobacillus reuteri CH53, cell growth, glycerol consumption, 1,3-propanediol production, lactic acid production, acetic acid production, and ethanol production were measured while each of the strains was fed-batch cultured. 3 L of MRS broth containing 20 g/L of glycerol, 10 g/L of proteose peptone NO. 3, 10 g/L of beef extract, 5 g/L of yeast extract, 20 g/L of glucose, 1 g/L of polysorbate 80, 2 g/L of ammonium citrate, 5 g/L of sodium acetate, 0.1 g/L of magnesium sulfate, 0.05 g/L of manganese sulfate, and 2 g/L dipotassium phosphate was placed in a 5-L fermenter, and 10% (v/v) of each strain culture with OD_{600 nm}=8. 0 was inoculated into the MRS broth and cultured at 100 rpm and 37° C. and without aeration. During culture, a mixture of 450 g/L of glucose and 450 g/L of glycerol were continuously fed as substrates. The absorbance (OD_{600 nm}) was measured while fed-batch culture was carried out. 1 mL of each culture was centrifuged at 13,000 rpm for 5 minutes, and then the supernatant was filtered through a 0.22-μm filter and the concentrations of glycerol, 1,3-propanediol, lactic acid, acetic acid and ethanol in the culture were measured using high-performance liquid chromatography. The concentrations were analyzed by high-performance liquid chromatography equipped with an ion exchange column (300×78 mm, Aminex HPX-87H, Bio-Rad) and a differential refraction detector (RID) using 0.25 mM H₂SO₄ as a mobile phase at a flow rate of 0.6 ml/min.

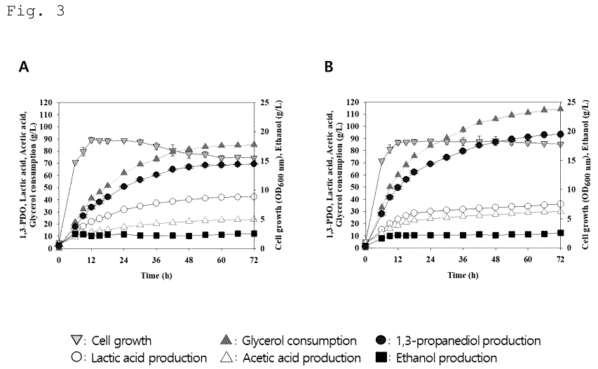

As a result, it was found that the parent strain Lactobacillus reuteri CH53 reached the death phase after 24 hours of culture, whereas cell growth of the Lactobacillus reuteri JH53 strain of the present invention did not decrease until 72 hours of culture, and lactic acid production of the Lactobacillus reuteri JH53 strain decreased compared to that of the parent strain Lactobacillus reuteri CH53. In addition, it was found that the parent strain Lactobacillus reuteri CH53 finally produced 69.2 g/L of 1,3-propanediol at 72 hours of culture, whereas the Lactobacillus reuteri JH53 strain of the present invention produced 93.2 g/L of 1,3-propanediol, indicating that 1,3-propanediol production of the Lactobacillus reuteri JH53 strain increased by about 34.6% compared to that of the parent strain (FIG. 3).

### [Accession Number]

Depository authority: the Korea Research Institute of Bioscience and Biotechnology

Accession number: KCTC14360BP

Deposit date: Nov. 9, 2020

1. A Lactobacillus reuteri JH53 strain (accession number: KCTC14360BP) with excellent organic acid resistance and conversion rate of glycerol to 1,3-propanediol.

2. The Lactobacillus reuteri JH53 strain according to claim 1, wherein the organic acid is lactic acid or acetic acid.

3. The Lactobacillus reuteri JH53 strain according to claim 1, wherein 1,3-propanediol production of the strain increased by 20 to 40% compared to that of a Lactobacillus reuteri CH53 strain (KCTC13149BP) as a parent strain.

4. A composition for producing 1,3-propanediol containing the strain according to claim 1 or a culture thereof as an active ingredient.

5. A cosmetic composition for moisturizing skin containing the strain according to claim 1 or a culture thereof as an active ingredient.

6. The cosmetic composition according to claim 5, further containing at least one auxiliary selected from among a fatty substance, an organic solvent, a solubilizer, a thickener, a gelling agent, a softener, an antioxidant, a suspending agent, a stabilizer, a foaming agent, a flavoring agent, a surfactant, water, an ionic or nonionic emulsifying agent, a filler, a metal ion sequestering agent, a chelating agent, a preservative, a vitamin, a blocking agent, a wetting agent, an essential oil, a dye, a pigment, a hydrophilic or lipophilic activator, and a lipid vesicle.

7. A method for producing 1,3-propanediol comprising a step of culturing the strain according to claim 1.
